(19) United States
(12) Patent Application Publication
(10) Pub. No.: US 2004/0063202 A1
(43) Pub. Date: Apr. 1, 2004

(54) Neurogenesis from hepatic stem cells

(76) Inventors: Bryon E. Petersen, Gainesville, FL (US); Jie Deng, Gainesville, FL (US)

Correspondence Address: Stanley A. Kim., Ph.D., Esq, Akerman Senterfitt, Suite 400, 222 Lakeview Avenue, West Palm Beach, FL, 33402-3188, US

(21) Appl. No.: 10/651,829

(22) Filed: Aug. 28, 2003

Related U.S. Application Data
(60) Provisional application No. 60/406,513, filed on Aug. 28, 2002.

(51) Int. Cl. C12N 5/08

(52) U.S. Cl. 435/368

(57) ABSTRACT

In vitro and in vivo approaches were used to induce hepatic oval cells to differentiate into cells expressing a neural cell-specific marker and displaying a neural morphology. Increasing cAMP in hepatic oval cells or co-culturing hepatic oval cells with neurospheres caused the hepatic oval cells to develop into cells displaying a neural cell-like phenotype. Hepatic oval cells transplanted into a brain differentiated into cells that phenotypically resembled all of the major cell types in the brain, including astrocytes, neurons, and microglia.

### CROSS-REFERENCE TO RELATED APPLICATIONS

The present application claims the priority of U.S. provisional patent application No. 60/406,513 filed on Aug. 28, 2002.

### STATEMENT AS TO FEDERALLY SPONSORED RESEARCH

This invention was made with United States government support under grant number DK-58614 and DK-60015 awarded by the National Institutes of Health. The United States government may have certain rights in the invention.

### FIELD OF THE INVENTION

The invention relates generally to the fields of developmental biology and medicine. More particularly, the invention relates to compositions and methods for producing a neuron-like cell from an hepatic oval cell (HOC).

### BACKGROUND

Neurodegenerative disorders such as Alzheimer's disease, Huntington's disease and Parkinson's disease are a heterogeneous group of diseases of the nervous system that have many different etiologies. A number are hereditary, some are secondary to toxic or metabolic processes, and some result from infections. Others have no known etiology. Neurodegenerative diseases are often age-associated, chronic, and progressive. Many also lack effective treatments. Neuropathologically, these diseases are characterized by abnormalities of relatively specific regions of the brain and populations of neurons. The clinical phenotype of the illnesses correlates with the particular cell groups involved. The prevalence, morbidity and mortality of neurodegenerative diseases result in significant medical, social, and financial burdens.

A variety of drugs have been developed to treat the symptoms of neurodegenerative diseases. In many cases, however, these drugs function by merely ameliorating symptoms of the disease rather than by restoring the patient to a healthy state. Methods for treating neurodegenerative diseases by replacing failed cells with new, undamaged cells would thus be therapeutically more preferable.

### SUMMARY

Methods and compositions for inducing the differentiation of an HOC into a neuron-like cell have been developed. In vitro and in vivo approaches were used to induce HOCs to differentiate into cells displaying a neural phenotype. HOCs transplantated into a brain in an animal differentiated into cells that phenotypically resembled all of the major cell types in the brain, including astrocytes, neurons, and microglia. This discovery should facilitate the practical implementation of cell replacement/regeneration as a method of treating neurodegenerative diseases because it provides a method to generate a sufficient supply of functional neural-like cells for transplantation. Moreover, applications of the invention that use autologous cells that have been differentiated into a neural-like cells as donors avoids rejection of the cells by the immune system.

Accordingly, the invention features a method for producing a cell that expresses a neural cell phenotype. The method includes the steps of: (a) providing an hepatic oval cell; and (b) placing the hepatic oval cell under conditions that promote the differentiation of the hepatic oval cell into a cell that expresses a neural cell phenotype. The neural cell phenotype can be expression of marker such as NFM, nestin, MAP2, βIII tubulin, α-internexin, GFAP, S100, and/or CD11b.

In one aspect of the invention, the step (b) of placing the hepatic oval cell under conditions that promote the differentiation of the hepatic oval cell into a cell that expresses a neural cell phenotype includes contacting the hepatic oval cell with an agent increases cAMP concentration (e.g., analogue of cAMP such as dibutyryl cAMP, or an inhibitor of cAMP phosphodiesterase such as 3-isobutyl-1-methylxanthine) in the hepatic oval cell.

In another aspect of the invention, the step (b) of placing the hepatic oval cell under conditions that promote the differentiation of the hepatic oval cell into a cell that expresses a neural cell phenotype includes culturing the hepatic oval cell with a neurosphere.

In yet another aspect of the invention, the step (b) of placing the hepatic oval cell under conditions that promote the differentiation of the hepatic oval cell into a cell that expresses a neural cell phenotype includes transplanting the hepatic oval into a central nervous system tissue (e.g., brain) in an animal.

Also within the invention is a cell made according to one of the foregoing methods. The cell can express a neural cell marker such as NFM, nestin, MAP2, βIII tubulin, α-internexin, GFAP, S100, and/or CD11b.

The invention further features a method of introducing a cell of the invention into a host animal subject. The method includes of the steps of providing the subject (e.g., a human patient suffering from a neurodegenerative disorder and introducing into the subject a cell of the invention.

When referring to a cell, the phrase “neural cell phenotype” means a characteristic generally expressed by one or more neural cells, but not generally expressed by non-neural cells. A neural cell phenotype can be expression of a neural cell-associated marker or a morphological characteristic.

By the term “neurosphere” is meant an aggregate or cluster of cells which includes neural stem cells and primitive progenitors. See, e.g., Reynolds & Weiss, (1992) Science 255, 1707-1710.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention provides compositions and methods for differentiating an HOC into a neural-like cell, that is a cell that phenotypically resembles a cell of the nervous system, e.g., a neuron, a microglial cell, or an astrocyte. In the experiments described below, HOCs were subjected to various in vivo and in vitro protocols that caused the cells to express neuronal cell-associated marker proteins (e.g., nestin, s100, MAP II, GFAP, βIII tubulin, s100, CD11b, NFN and α-internexin) and/or to develop a neural cell-like morphology, e.g., elongation or establishment of neuron-like cell processes. The below described preferred embodiments illustrate adaptations of these compositions and methods. Nonetheless, from the description of these embodiments, other aspects of the invention can be made and/or practiced based on the description provided below.

### Biological Methods

Methods involving conventional biological, cell culture, immunological and molecular biological techniques are described herein. Such techniques are generally known in the art and are described in detail in methodology treatises. Cell culture techniques are generally known in the art and are described in detail in methodology treatises such as Culture of Animal Cells: A Manual of Basic Technique, 4th edition, by R. Ian Freshney, Wiley-Liss, Hoboken, N.J., 2000; and General Techniques of Cell Culture, by Maureen A. Harrison and Ian F. Rae, Cambridge University Press, Cambridge, UK, 1994. Immunological methods (e.g., preparation of antigen-specific antibodies, immunoprecipitation and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Molecular biological techniques are described in references such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates).

### Hepatic Oval Cells

Methods of the invention utilize HOCs as source cells from which cells having a neural cell-like phenotype can be made. HOCs can be derived from the liver of any animal known to contain such cells, e.g., rodents such as rats and mice, and primates such as human beings. A variety of methods for obtaining HOCs suitable for use in the invention is known. Any one of these might be might be used.

In general, HOCs may be obtained from a liver that (1) has been damaged and (2) prevented from regenerating. As an example of a specific protocol, HOC activation, proliferation, and differentiation can be induced in rats by a two-step procedure. In the first step, the animals are exposed to 2-acetylaminofluorene (2-AAF) to suppress hepatocyte proliferation. In the second step, liver injury is induced by either partial hepatectomy or by treatment with carbon tetrachloride. Petersen, et al., Hepatology 27, 1030-1038 (1998). As another example, a large number of HOCs can be induced in mice by adding the chemical 3,5-diethoxycarbonyl-1,4-dihydrocollidin (DDC) at a 0.1% concentration to the animals' normal chow. Preisegger et al., Lab. Invest. 79:103, 1999. HOCs can be isolated from animals by known techniques, e.g., a two-step liver perfusion method as described by Selgen et al. (J. Toxic. Environ. Health 5:551, 1979).

Because one aspect the invention relates to transplantation into humans, a preferred source of mammalian HOCs is human liver. HOCs from humans can be obtained, for example, by core biopsy of the liver. Following dispersion of the liver cells using enzymes such as trypsin and collagenase, primary cultures can be established according to published techniques. Upon prolonged culturing, the proliferating oval cells can be clonally expanded. Other methods for obtaining human hepatic oval (or stem-like) cells are described in, e.g., published U.S. patent applications 20020182188 to Reid et al. and 20010024824 to Moss et al.

### HOC Isolation

HOCs can be purified from liver based on their expression of certain cell surface markers. HOCs are known to express high levels of surface Thy-1, cytokeratin (CK)-19, OC.2 and OV6, as well as cytoplasmic alpha-fetoprotein (AFP) and gamma-glutamyl-transpeptidase (GGT) (Dabeva, et al. Proc. Natl. Acad. Sci. U.S. A. 94:7356-7361, 1997; Lemire et al., Am. J. Pathol. 139: 535-552, 1991; Petersen, et al., Hepatology 27: 433-445, 1998; Shiojiri et al., Cancer Res. 51: 2611-2620, 1991). Murine hepatic oval cells can be selected on the basis of their expression of Sca-1. See, Petersen et al., J. Hepatology, 37:632, 2003. In an similar manner, human hepatic oval cells can be selected on the basis of their expression of c-kit, pi class glutathione S-transferase, and CK-18 and CK-19.

A population of cells containing a cell expressing a HOC-selective marker is contacted with an antibody that binds specifically to the marker. Once marker-positive cells are bound by antibody, such cells may then be isolated by any number of well-known immunosorting/immunoseparating methods including FACS. Other methods of separation can also be used such as MACS, immunopanning or selection after transfection with a promoter that drives a marker gene. Immunomagnetic separation/sorting techniques generally involve incubating cells with a primary antibody specific to a surface antigen found on the target cell type, immunologically coupling the target cells to magnetic beads (e.g., marker-specific antibody conjugated to magnetic particles), and then separating the target cells out from the heterogeneous cell population using a magnetic field.

Immunopanning techniques involve the plating of a tissue culture dish with an antibody that binds the cell marker of interest, plating of cells onto the dish, washing away unbound cells, and isolating the antibody-bound target cells by trypsin digest. Immunopanning techniques are well known in the art and are described in Mi and Barres J. Neurosci. 19:1049-1061, 1999; Ben-Hur et al., The Journal of Neuroscience 18:5777-5788, 1998; Ingraham et al., Brain Res Dev Brain Res 112:79-87, 1999; Murakami et al., J. Neurosci. Res. 55:382-393, 1999; and Oreffo et al., J. Cell Physiol. 186:201-209, 2001.

Additionally, combinations of immunosorting/immunoseparating methods can be used to isolate a cell that expresses a neural cell-specific marker from a population of cells. For example, magnetic microbead selection can be followed by an immunoadsorption technique (e.g., biotinylated antibody applied to a column of avidin-coated sephadex beads or an immunoaffinity column, Johnsen et al., Bone Marrow Transplant 24:1329-1336, 1999; Langet al., Bone Marrow Transplant 24:583-589, 1999; Handgretinger et al., Bone Marrow Transplant 21:987-993, 1998). Another example of a sorting technique involves use of a magnetic cell sorter followed by a selection step with an anti-marker antibody bound to immunomagnetic beads (Martin-Henao et al., Transfusion 42:912-920, 2002). A combination of two MACS systems may also be used in methods of the invention (Lang et al., Bone Marrow Transplant 24:583-589, 1999).

For example, fluorescence-activated cell sorting (FACS) can be used to isolate Thy-1⁺ hepatic oval stem cells from carbon tetrachloride-injured rat livers treated with 2-AAF (to block hepatocyte regeneration) with a purity of >95%. Petersen et al., Hepatology 27, 1030-1038 (1998). As another example, wild-type Sca-1+ and Sca-1-murine oval cells, obtained from MACs magnetic sorting, are incubated with fluorescein isothiocyanate conjugated (FITC-) anti-Sca-1 and FITC-anti-rat IgG2a antibodies (PharMingen; 1:500) for 30 min at room temperature. Cells are then pelleted by centrifugation at 200 g and washed twice in PBS to eliminate unbound antibodies. Approximately 10⁶ cells/ml cell suspension is run through a flow cytometer (CELLQuest, Becton Dickinson FACScan).

### Induction Of Differentiation

HOC can be induced to differentiate into cells with a neural cell-like phenotype by culturing the cells in an appropriate in vitro or an in vivo environment. As an example of the former, HOCs cultured in vitro in culture medium containing high levels of an agent that increases cellular cAMP levels (e.g., 1 mM dibutyryl cAMP; dbcAMP) differentiate into a neural cell-like cells. Similarly, HOCs cultured in vitro in culture medium containing an inhibitor of cAMP phosphodiesterase (e.g., 3-isobutyl-1-methylxanthine; IBMX) differentiate into a neural-like cells. In another in vitro method, HOCs are co-cultured with neurospheres (cultured neural cells derived from trypsinized neo-natal mouse brains; NS) to induce their differentiation into a cells exhibiting a neural cell-like phenotype.

In vivo procedures can also lead to trans-differentiation of HOCs cells into cells displaying a neural cell-like phenotype. For example, HOCs injected directly into the brain of a living animal differentiate in situ into cells with a neural cell-like phenotype.

HOC differentiation into a cell displaying a neural phenotype can be assessed by any available method of distinguishing different cell types, e.g., based on cell morphology or expression of particular markers. For example, microscopy can be used to determine if HOCs change into cells that more closely resemble a neural cell. Expression of neural cell differentiation markers such as nestin, s100, Map II, glial fibrillary acid protein (GFAP), βIII tubulin, s100, CD11b, neurofilament associated protein medium subunit (NFM) and α-internexin also indicates that an HOC has differentiated into a neural-like cell.

### Isolating Cells Expressing a Neural Cell-Specific Marker

HOCs differentiated into cells with a neural cell phenotype can be purified, e.g., for transplantation, from in vitro cultures or animal tissues using conventional techniques. For example, a population of cells suspected of containing a cell expressing a neural cell-specific marker is contacted with an antibody that binds specifically to the marker. Once marker-positive cells are bound by antibody, such cells may then be isolated by any number of well-known immunosorting/immunoseparating methods including FACS, MACS, immunopanning or selection after transfection with a promoter that drives a marker gene.

### Administration of Cells

Neural-like cells differentiated from HOC can be administered to an animal (e.g., a human subject suffering from a neurodegenerative disease) by conventional techniques. For example, trans-differentiated neuron-like cells may be administered directly to a target site (e.g., a brain) by, for example, injection (of cells in a suitable carrier or diluent such as a buffered salt solution) or surgical delivery to an internal or external target site (e.g., a ventricle of the brain), or by catheter to a site accessible by a blood vessel. For exact placement, the cells may be precisely delivered into brain sites by using stereotactic injection techniques. For example, the mammalian subject to be treated can be placed within a stereotactic frame base that is MRI-compatible and then imaged using high resolution MRI to determine the three-dimensional positioning of the particular site being treated. According to this technique, the MRI images are then transferred to a computer having the appropriate stereotactic software, and a number of images are used to determine a target site and trajectory for delivery of the cells. Using such software, the trajectory is translated into three-dimensional coordinates appropriate for the stereotactic frame. For intracranial delivery, the skull will be exposed, burr holes will be drilled above the entry site, and the stereotactic apparatus positioned with the needle implanted at a predetermined depth. The cells can then be injected into the target site(s).

### Effective Doses

The cells described above are preferably administered to a mammal in an effective amount, that is, an amount capable of producing a desirable result in a treated subject (e.g., reversing symptoms of a neurodegenerative disease in the subject). Such therapeutically effective amounts can be determined empirically. Although the range may vary considerably, a therapeutically effective amount is expected to be in the range of 1×10⁶ to 1×10¹⁰ cells/animal.

### EXAMPLES

The present invention is further illustrated by the following specific examples. The examples are provided for illustration only and should be construed as limiting the scope of the invention in any way.

### Example 1

### In Vitro Trans-Differentiation

HOCs acquire characteristics of a neuron-like cell phenotype when treated with IBMX and dbcAMP, both of which elevate the level of cytoplasmic cAMP. One day before the experiment began, HOCs were transplanted into a 6 well plate at 60% confluence, and cultured overnight in Medium A, a medium that contained IMEM, supplemented with 10% FBS, 1% insulin, 10 ng/ml IL-3, 10 ng/ml IL-6, 10 ng/ml SCF, and 1000 U/ml LIF. On day two, the culture media was replaced with induction media (Medium A lacking LIF but supplemented with 0.5 mM IBMX, 1 mM dbcAMP without LIF). Cells were then cultured for up to four weeks in a humidified 37° C., 5% CO₂ incubator, during which time, the media was changed once per week. Cells in the culture started to send out processes 24 hours after being added to the induction medium. After about one week, 30% of the cells exhibited neuron-like cell morphology.

Cells in the culture were later examined for expression of neural cell differentiation markers. After four weeks in the induction medium culture, the cells were removed from the culture and fixed for 5 minutes with 4% paraformaldehyde. After washing with PBS 3 times for 5 minutes and blocking in 10% goat serum for 30 minutes, primary antibodies against a neuron-specific protein (βIII tubulin) and an astrocyte-specific protein (S100) were then incubated with the cells for 1 hour at room temperature. After washing the cells again in PBS 3 times for 5 minutes per wash, the cells were incubated with fluorescent secondary antibodies for 1 hour at room temperature. The cells were then washed 3 times for 5 minutes per wash in PBS, placed on a cover-slip, and subjected to fluorescent microscopy. Most of the cells in the culture were S100 positive; a small population of the cells were βIII tubulin positive.

### Example 2

### Co-Culture Trans-Differentiation

HOCs acquired the characteristics of a neuron-like cell phenotype when co-cultured with neural cells differentiated from neurospheres (NS). NS were generated from postnatal day 5-7 mouse brains. Briefly, pups were decapitated under deep anesthesia (intraperitoneal injection of sodium phenobarbital), and their brains were removed. After removing the olfactory bulbs and the cerebellum, brain tissue was cut into small pieces, washed in PBS and trypsinized at 37° C. for 10 minutes to dissociate the cells. After further washing, the cells were re-suspended in 2% methyl cellulose dissolved in DMEM/F12 supplemented with N2 and a growth factor cocktail of 10 ng/ml basic FGF and 20 ng/ml EGF. Cells were then transferred to culture dishes coated with anti-adhesives. After about two weeks in culture, NS of about 150 μm in diameter were harvested and laid on cover slips coated with laminin/polyornithine in DMEM/F12 supplemented with N2. This procedure induced trans-differentiation. To label the HOCs for the co-culture system, rat HOCs were transfected with lentiviral vectors carrying a GFP gene. The GFP+ HOCs were placed on the neural cell layers growing out from the NS and cultured for up to 4 weeks. Many of the HOCs changed into an elongated morphology after about 3 days of co-culturing and after 4 weeks of co-culture, some GFP+ HOC appeared positive for βIII tubulin and α-internexin as determined by immunostaining.

### Example 3

### In Vivo Transdifferentiation

Hepatic oval cell induction and enrichment from mouse liver. According to the protocol established by Preisseger et al., (Lab. Invest. 79:103, 1999), adult C57BL6/GFP+/+ transgenic mice were fed a normal diet supplemented with 0.1% DDC (BioServe, Frenchtown, N.J.) for 6 weeks. To isolate HOCs, a two-step liver perfusion was performed as described by Selgen et al. (J. Toxicol. Environ. Health, 5:551, 1979), collecting the nonparenchyma fraction (NPC) using gradient centrifugation. The NPC was incubated with Sca-1 antibody conjugated to micromagnetic beads, and the cell suspension was processed through magnetic columns to enrich the oval cell population positive for Sca-1 (MACs, Miltenyi Biotec).

FACs analysis for purity on MACs-sorted Sca-1+ oval cells. Wild-type Sca-1+ and Sca-1− oval cells, obtained from MACs magnetic sorting, were incubated with fluorescein isothiocyanate (FITC)-Sca-1 and FITC-rat IgG2a antibodies (PharMingen; 1:500) for 30 min at room temperature. Cells were then pelleted by centrifugation at 200 g and washed twice in PBS to eliminate unbound antibodies. Approximately 10⁶ cells/ml cell suspension was run through a flow cytometer (CELLQuest, Becton Dickinson FACScan).

Immunocytochemistry of MACs-sorted oval cells. Wild-type Sca-1+ oval cells, obtained from a MACs magnetic cell sorter, were cytocentrifuged to slides, fixed with 4% paraformaldehyde in PBS, and examined for mouse oval cell markers as described in Petersen et al., Hepatology 27:433-445, 1998. A6 antibody (a gift from Dr. Valentina Factor of the NIH;

1:20) and anti-fetal protein (AFP; Santa Cruz Biotechnology; 1:200) were used for the immunocharacterization of oval cells.

Culture of mouse oval cells. Approximately 10⁶ Sca-1+ mouse oval cells, obtained from MACs cell sorting were cultured in a 35-mm culture dish (Costar, Corning) in HOC culture media (89% Iscove's modified Dulbecco's medium, 10% FBS, 1% insulin, 1000 U/ml of leukemia inhibitory factor, 20 ng/ml granulocyte macrophage colony stimulating factor, and 100 ng/ml each of stem cell factor, interleukin-3, and interleukin-6).

Cell transplantation into neonatal mouse brain. Sca-1+ MACs-sorted primary dissociates of GFP+ oval cells were transplanted into the lateral ventricle of postnatal day 1 wild-type C57BL6 mice within the first 24 h after birth. Newborn pups were anesthetized by hypothermia and placed in a clay mold. The head was transilluminated under a dissection microscope, and a Hamilton syringe with a beveled tip was lowered through the scalp and skull immediately anterior to bregma. Approximately 2.5×10⁵ GFP+ HOCs in 1 μl volume of Dulbecco's modified Eagle's medium/F12 (DMEM/F12, Gibco) were then slowly pressure injected into the left lateral ventricle. Immediately after injection, pups were warmed in a 37° C. incubator, and returned to the mother after approximately 30 min. At 10 days post-transplantation, mice were euthanized with an overdose of Avertin and perfused transcardially with 4% paraformaldehyde in PBS. The brain tissue was excised, post-fixed overnight in perfusate, and sectioned through the coronal plane into 40-μm slices with a vibratome.

In vivo phagocytosis assay. An in vivo phagocytosis assay of microglia was performed by adding fluorescent latex microbeads to the graft bolus immediately prior to transplantation. Latex microbeads (Sigma L-0530; 0.5-m in diam; fluorescent blue conjugated) were added into the cell suspension (˜2.5×10⁵ cells/μl in DMEM/F12) at a concentration of 15% (0.15 μl bead solution/0.85 μl cell suspension). One microliter of cell/bead mixture was injected into the lateral ventricle of newborn pup brains as described above. Hosts were then allowed to survive for 10 days before the brains were fixed and processed for immunocharacterization.

Immunolabeling of brain sections. Forebrains were cut with a vibratome into 40-m coronal sections exhaustively and processed free-floating for immunofluorescence. After blocking in PBS with 10% goat serum, sections were incubated overnight at 4° C. in primary antibodies directed against the following proteins: nestin, a marker of neuronal stem and progenitor cells (Developmental Studies Hybridoma Bank, University of Iowa; 1:250); the astrocyte-specific markers glial fibrillary acidic protein (GFAP; from Gerry Shaw, University of Florida; 1:200) and S1:200 (Sigma; 1:250); the microglia marker CD11b (Serotec; 1:200); and the neuronal markers neurofilament medium subunit (NFM; from Gerry Shaw, University of Florida; 1:500), alpha-internexin (α-IN; from Gerry Shaw, University of Florida; 1:200), and MAP2ab (Sigma; 1:500). The tissues were then washed in PBS, followed by incubation in appropriate secondary antibodies conjugated to R-phycoerythrin (R-PE) (Molecular Probes) at room temperature for 1 h. After a final wash in PBS, brain slices were mounted onto glass slides, viewed, and counted with a fluorescence microscope.

Quantification of grafted cells. Cell counting was performed under a fluorescence microscope (Olympus B×51). Every sixth section through the forebrain was selected for counting of grafted cells. A cell was counted if the cell body could be identified. Total number of cells was then obtained by multiplying the counted result by a factor of six. The standard deviations were obtained using Microsoft Office Excel statistic software.

To verify the purity obtained with the sorting method, FACs analysis was performed on MACs sorted Sca-1+ cells. After MACs sorting, only 20% of the Sca-1 epitopes were occupied by the Sca-1-conjugated magnetic beads, which allowed use of the remaining epitopes to perform the FACs analysis for purity. Histograms of the FACs analysis showed a distinct population of cells. MACs-sorted cells were over 90% positive for Sca-1 antibody, while the flow-through cells were Sca-1 negative. Immunocytochemistry was performed to verify that the Sca-1+ cells isolated by MACS were indeed oval cells. Immunocytochemistry revealed that the Sca-1+, MACs-sorted cells were also positive for A6 and AFP, known markers for mouse oval cells. When cultured in vitro, HOCs started to proliferate in about 5 days and formed colonies after about 2 weeks. The HOCs in culture appeared to be a homogeneous and undifferentiated cell population.

Ten days after transplantation of HOCs, intensely fluorescent GFP+ cells were seen within the host brain. The majority of surviving donor cells were located in periventricular areas in all of the mice with successful cell delivery. GFP+ cells were most frequently observed superficially along the walls of the lateral ventricle, but numerous grafted cells were also found to migrate laterally within the white matter of the corpus callosum. At points along the ventricular wall, grafted cells penetrated into the parenchyma of the brain, a phenomenon previously described following intraventricular transplantation of multipotent astrocytes (Zheng et al., 2002). The survival rate of the transplanted HOCs averaged 0.56+/−0.36% (n=9) of the total injected cells (Table 1). Approximately 11.5+/−2.5% (n=3) of grafted cells remained undifferentiated and were characterized by a small, rounded, non-process-bearing morphology. The remainder displayed varying degrees of differentiation and process extension. Seven of 36 animals receiving transplants did not contain any detectable donor cells.

TABLE I
Survival rate of transplanted HOCs in the neonatal mouse brain
Animal

| | No. of injected cells | No. of GFP+ | Percentage of survival |
|---|---|---|---|
| Animal No. | (× 10⁵) | cells | (%) |
| 5.3 | 2.5 | 680 | 0.27 |
| 13.1 | 2.5 | 390 | 0.16 |
| 14.6 | 2.5 | 2250 | 0.90 |
| 14.7 | 2.5 | 468 | 0.19 |
| 15.4 | 2.0 | 2022 | 1.01 |
| 15.5 | 2.0 | 1962 | 0.98 |
| 15.6 | 2.0 | 1302 | 0.65 |
| 15.7 | 2.0 | 1584 | 0.79 |
| 15.9 | 2.0 | 546 | 0.27 |
| Average | 2.2 | 1245 | 0.56 |

Note:
Nine mouse brains were counted. The GFP+ cells of every sixth section of the forebrain were counted for each brain. The total numbers of survived HOCs were obtained by multiplying the counted results by a factor of six. The standard deviation is 0.36%.

### Example 6

### Grafted Hepatic Oval Cells Express Neural Antigens

Differentiated GFP+ HOCs expressed neural-specific proteins in the neonatal mouse brain. The filament protein nestin has frequently been considered indicative of neural progenitor cells (Lendahl et al., 1990). It was found that 22.1+/−11.6% (n=4) of surviving donor cells were immunopositive for nestin (Table 2), suggesting that HOCs may be able to assume the phenotype of early neural lineage. Of the donor cells that differentiated, the majority exhibited a typical amoeboid or ramified microglia morphology. A smaller fraction displayed the stellate, process-rich characteristics of astrocyte morphology. Immunolabeling with the Mac-1 antibody, directed against the CD11b epitope characteristic of macrophages, showed that 60.6+/−10.5% (n=3) of the GFP+ donor cells expressed this microglial marker (Table 2). Additionally, 34.7+/−9.0% (n=4) and 27.2+/−5.7 (n=3) of donor cells expressed the astrocyte-specific proteins GFAP and S100, respectively (Table 2). Many of the cells expressing astrocyte proteins were located within the corpus callosum, and their processes could be seen intertwining with the processes of native astrocytes. A small number of donor cells were also seen to be immunopositive for neuron specific markers. The neuronal marker NF-M was expressed in 6.5+/−1.3% (n=3) of the grafted cells (Table 2), and a comparable number expressed α-IN. A considerably larger percentage, 19.9+/−2.5% (n=3), of donor cells were immunopositive for MAP2 (Table 2).

TABLE 2
Composition of the neural markers in the transplanted HOCs in the
neonatal mouse brain

| | | No. of | No. of | Percentage of |
|---|---|---|---|---|
| | No. of | positive | GFP+ | positive cells |
| Markers | animals | cells | cells | (%) |
| GFAP | 4 | 78.8 | 227.0 | 34.7 +/− 9.0 |
| S100 | 3 | 68.0 | 250.0 | 27.2 +/− 5.7 |
| Mac1 | 3 | 102.7 | 169.3 | 60.6 +/− 10.5 |
| NFM | 3 | 11.0 | 168.0 | 6.5 +/− 1.3 |
| Nestin | 4 | 25.5 | 115.3 | 22.1 +/− 11.6 |
| Map2 | 3 | 55.0 | 276.0 | 19.0 +/− 2.5 |

Note:
Every sixth section of the forebrain was counted for each animal. The average numbers of cells positive for each marker, and the GFP+ cells, as well as the percentages of the number of positive cells among the total GFP+ cells (mean +/− SD) among all the mice inspected are shown.

Grafted cells with the antigenic profile of microglia also displayed appropriate phagocytic activity, since cotransplanted fluorescent microbeads were incorporated into their cytoplasm at high efficiency (Table 3). Microbeads were incorporated in 58.7% of grafted GFP+ cells, as well as numerous indigenous microglia, and these cells were subsequently shown to express the CD11b antigen, characteristic of macrophages, including brain microglia. GFP expression of oval cells colocalized with immunostaining with Mac1 antibody against CD11b. Many Mac1+ oval cells coexisted with native microglias.

TABLE 3
Percentage of GFP+ cells taking up
microbeads among the total GFP+ cells

| Animal No. | GFP+ with beads | Total GFP+ | GFP+ with beads (%) |
|---|---|---|---|
| 21.5 | 52 | 78 | 66.7 |
| 21.6 | 23 | 37 | 62.2 |
| 21.7 | 14 | 25 | 56.0 |
| 21.8 | 14 | 28 | 50.0 |
| Average | 26 | 42 | 58.7 |

Note:
Every sixth section of the forebrain was inspected for each animal. The standard deviation is 7.3% among all four mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for producing a cell that expresses a neural cell phenotype, the method comprising the steps of:
(a) providing an hepatic oval cell; and
(b) placing the hepatic oval cell under conditions that promote the differentiation of the hepatic oval cell into a cell that expresses a neural cell phenotype.

2. The method of claim 1, wherein the neural cell phenotype comprises expression of marker selected from the group consisting of: NFM, nestin, MAP2, βIII tubulin, α-internexin, GFAP, S100, and CD11b.

3. The method of claim 1, wherein step (b) comprises contacting the hepatic oval cell with an agent increases cAMP concentration in the hepatic oval cell.

4. The method of claim 3, wherein the agent is an analogue of cAMP.

5. The analogue of claim 4, wherein the analogue is dibutyryl cAMP.

6. The method of claim 3, wherein the agent is an inhibitor of cAMP phosphodiesterase.

7. The method of claim 6, wherein the agent is 3-isobutyl-1-methylxanthine.

8. The method of claim 1, wherein step (b) comprises culturing the hepatic oval cell with a neurosphere.

9. The method of claim 1, wherein step (b) comprises transplanting the hepatic oval into a central nervous system tissue in an animal.

10. The method of claim 9, wherein the central nervous system tissue is a brain.

11. A cell that expresses a neural cell phenotype, the cell being made according to the method of claim 1.

12. The cell of claim 11, wherein the cell expresses are marker selected from the group consisting of: NFM, nestin, MAP2, βIII tubulin, α-internexin, GFAP, S100, and CD11b.

13. The cell of claim 11, wherein the marker is NFM.

14. The cell of claim 1, wherein the marker is nestin.

15. The cell of claim 11, wherein the marker is MAP2.

16. The cell of claim 11, wherein the marker is βIII tubulin.

17. The cell of claim 11, wherein the marker is α-internexin.

18. The cell of claim 11, wherein the marker is GFAP.

19. The cell of claim 11, wherein the marker is S100.

20. The cell of claim 11, wherein the marker is CD11b.

21. A method of introducing a cell into a host animal subject, the method comprising the steps of:
(a) providing the animal subject; and
(b) introducing into the subject a cell made according to the method of claim 1.
